(19) United States
(12) Patent Application Publication
(10) Pub. No.: US 2014/0249141 A1
(43) Pub. Date: Sep. 4, 2014

(54) 1,4-DIAZABICYCLO[3.2.2]NONANES AS NEURONAL NICOTINIC ACETYLCHOLINE RECEPTOR LIGANDS

(71) Applicants: Anatoly Mazurov, Greensboro, NC (US); Lan Miao, Advance, NC (US); Todd Showalter, Clemmons, NC (US)

(72) Inventors: Anatoly Mazurov, Greensboro, NC (US); Lan Miao, Advance, NC (US); Todd Showalter, Clemmons, NC (US)

(73) Assignee: Targacept, Inc., Winston-Salem, NC (US)

(21) Appl. No.: 14/239,817

(22) PCT Filed: Aug. 20, 2012
(86) PCT No.: PCT/US12/51518

§ 371 Date: Apr. 30, 2014

Related U.S. Application Data
(60) Provisional application No. 61/526,181, filed on Aug. 22, 2011.

(51) Int. Cl. C07D 519/00; A61K 31/551

(52) U.S. Cl. CPC C07D 519/00; A61K 31/551
USPC 514/221; 540/556

(57) ABSTRACT

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including inflammatory diseases and diseases associated with dysfunction of the central nervous system (CNS).

### FIELD OF THE INVENTION

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several reviews. See, for example, Arneric et al., Biochem. Pharmacol. 74: 1092 (2007), Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002), Yang et al., Acta Pharmacol. Sin. 30(6): 740-751 (2009). Among the kinds of indications for which NNR ligands have been proposed as therapies are cognitive disorders, including Alzheimer's disease, attention deficit disorder, and schizophrenia (Biton et al., Neuropsychopharm. 32: 1 (2007), Boess et al., J. Pharmacol. Exp. Ther. 321: 716 (2007), Hajos et al., J. Pharmacol. Exp. Ther. 312: 1213 (2005), Newhouse et al., Curr. Opin. Pharmacol. 4: 36 (2004), Levin and Rezvani, Curr. Drug Targets: CNS Neurol. Disord. 1: 423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Curr. Drug Targets: CNS Neurol. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Bordia et al., J Pharmacol. Exp. Ther. 327: 239 (2008), Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001)).

There exists a heterogeneous distribution of nAChR subtypes in both the central and peripheral nervous systems. For instance, the α4β2, α6 containing, α7, and α3β2 subtypes are predominant in vertebrate brain, whereas the α3β4 subtype is predominate at the autonomic ganglia, and the α1β1δγ and α1β1δε subtypes are predominant at the neuromuscular junction (see Dwoskin et al., Exp. Opin. Ther. Patents 10: 1561 (2000) and Holliday et al. J. Med. Chem. 40(26), 4169 (1997)). Compounds which selectively target the CNS predominant subtypes have potential utility in treating various CNS disorders. However, a limitation of some nicotinic compounds is that they lack the selectivity required to preferentially target CNS receptors over receptor located in the muscle and ganglion. Such drugs are often associated with various undesirable side effects. Therefore, there is a need to have compounds, compositions, and methods for preventing or treating various conditions or disorders where the compounds exhibit a high enough degree of nAChR subtype specificity to elicit a beneficial effect, without significantly affecting those receptor subtypes which have the potential to induce undesirable side effects, including, for example, appreciable activity at cardiovascular and skeletal muscle sites.

### SUMMARY OF THE INVENTION

The present invention includes compounds which bind with high affinity to NNRs, preferably of the α7 subtype. The present invention also relates to pharmaceutically acceptable salts prepared from these compounds.

The present invention includes compounds of Formula I:

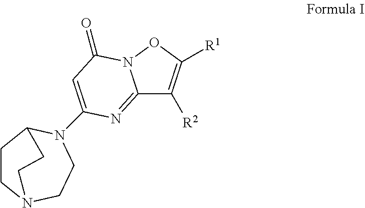

wherein:

each of R¹ and R² individually is H, C₁₋₆ alkyl, aryl, or aryl-substituted C₁₋₆ alkyl, or

R¹ and R² combine with the carbon atoms to which they are attached to form a 5- or 6-membered carbocyclic ring, either aromatic or non-aromatic,

or a pharmaceutically acceptable salt thereof.

The present invention includes pharmaceutical compositions comprising a compound of the present invention or a pharmaceutically acceptable salt thereof. The pharmaceutical compositions of the present invention can be used for treating or preventing a wide variety of conditions or disorders, particularly those disorders mediated by nicotinic acetylcholine receptors, more particularly those mediated by the α7 subtype, more particularly age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction. Thus, the present invention includes a method for treating, delaying the onset of, or slowing the progression of such disorders in mammals in need of such treatment. The methods involve administering to a subject a therapeutically effective amount of a compound of the present invention, including a salt thereof, or a pharmaceutical composition that includes such compounds.

### BRIEF DESCRIPTION OF THE FIGURES

FIGS. 1 and 2 illustrate the effects of the compounds of the present invention in providing a considerable reduction in airway hyperresponsiveness as demonstrated through the ovalbumin-induced allergic asthma model, a widely used model to reproduce the airway eosinophilia, pulmonary inflammation, and elevated IgE levels found during asthma and similar conditions and disorders such as COPD, rhinitis, and the like.

FIG. 1 illustrates Compound A reduces methacholine (MCh)-induced bronchoconstriction in ova-challenged mice. Penh is an index of airway resistance. Asterisks indicate P>0.05 compared to control.

FIG. 2 provides an illustration by a percentage change in Penh. Again, asterisks indicate P>0.05 compared to control.

### DETAILED DESCRIPTION OF THE INVENTION

### I. Compounds

The present invention includes compounds of Formula I:

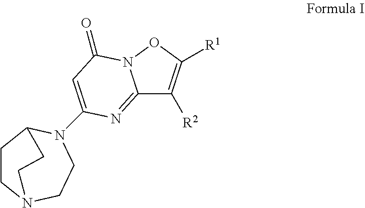

wherein

each of R¹ and R² individually is H, C₁₋₆ alkyl, aryl, or aryl-substituted C₁₋₆ alkyl, or

R¹ and R² combine with the carbon atoms to which they are attached to form a 5- or 6-membered carbocyclic ring, either aromatic or non-aromatic;

or a pharmaceutically acceptable salt thereof.

In one embodiment, a compound is selected from the group consisting of:
5-(1,4-diazabicyclo[3.2.2]nonan-4-yl)-2-methyl-7H-isoxazolo[2,3-a]pyrimidin-7-one,
5-(1,4-diazabicyclo[3.2.2]nonan-4-yl)-2-ethyl-7H-isoxazolo[2,3-a]pyrimidin-7-one,
5-(1,4-diazabicyclo[3.2.2]nonan-4-yl)-2-benzyl-7H-isoxazolo[2,3-a]pyrimidin-7-one,
5-(1,4-diazabicyclo[3.2.2]non-4-yl)-2-phenyl-7H-isoxazolo[2,3-a]pyrimidin-7-one,
2-(1,4-diazabicyclo[3.2.2]non-4-yl)-4H-pyrimido[1,2-b][1,2]benzoxazol-4-one,

or a pharmaceutically acceptable salt thereof.

In one embodiment, the present invention is compound 5-(1,4-diazabicyclo[3.2.2]nonan-4-yl)-2-methyl-7H-isoxazolo[2,3-a]pyrimidin-7-one or a pharmaceutically acceptable salt thereof. This compound may also be referred to as Compound A.

One aspect of the present invention includes a pharmaceutical composition comprising a compound of the present invention and a pharmaceutically acceptable carrier.

One aspect of the present invention includes a method for the treatment or prevention of a disease or condition mediated by neuronal nicotinic receptors comprising the administration of a compound of the present invention. In one embodiment, the neuronal nicotinic receptors are of the α7 subtype. In a further embodiment, the disease or condition is age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating, or sexual dysfunction.

One aspect of the present invention includes use of a compound of the present invention for the preparation of a medicament for the treatment or prevention of a disease or condition mediated by neuronal nicotinic receptors comprising the administration of a compound of the present invention. In one embodiment, the neuronal nicotinic receptors are of the α7 subtype. In a further embodiment, the disease or condition is age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

One aspect of the present invention includes a compound of the present invention for use as an active therapeutic substance. One aspect, thus, includes a compound of the present invention for use in the treatment or prevention of a disease or condition mediated by neuronal nicotinic receptors comprising the administration of a compound of the present invention. In one embodiment, the neuronal nicotinic receptors are of the α7 subtype. In a further embodiment, the disease or condition is age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

The scope of the present invention includes all combinations of aspects and embodiments.

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein, the term “cycloalkyl” refers to a fully saturated optionally substituted monocyclic, bicyclic, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

As used herein, the term “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, and tetrahydrothiophene.

As used herein, the term “aryl” refers to a single benzene ring or fused benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “heteroaryl” refers to a monocyclic five to seven membered aromatic ring, or to a fused bicyclic aromatic ring system comprising two of such aromatic rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein, multiple degrees of substitution includes substitution with one or more alkyl, halo, haloalkyl, alkoxy, alkylthio, aryloxy, arylthio, —NR^{a}R^{b}, —C(═O)NR^{a}R^{b}, —NR^{a}C(═O)R^{b}, —C(═O)R^{a}, —C(═O)OR^{a}, —OC(═O)R^{a}, —O(CR^{a}R^{b})₁₋₆C(═O)R^{a}, O(CR^{a}R^{b})_{d}NR^{b}C(═O)R^{a}, O(CR^{a}R^{b})₁₋₆NR^{b}SO₂R^{a}, —OC(═O)NR^{a}R^{b}, —NR^{a}C(═O)OR^{b}, —SO₂R^{a}, —SO₂NR^{a}R^{b}, or —NR²SO₂R³; where each R^{a} and R^{b} individually is hydrogen, alkyl, cycloalkyl, heterocyclyl, aryl, or arylalkyl, or R^{a} and R^{b} can combine with the atoms to which they are attached to form a 3- to 10-membered ring. Thus, as one example, Cy may be pyridinyl which may be substituted first by a halogen, such as F, and second by an alkoxy, such as —OCH₃.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as herein defined. Likewise, the term “alkylthio” refers to a group —SR^{a}, where R^{a} is alkyl as herein defined.

As used herein the term “aryloxy” refers to a group —OR^{a}, where R^{a} is aryl as herein defined. Likewise, the term “arylthio” refers to a group —SR^{a}, where R^{a} is aryl as herein defined.

As used herein “amino” refers to a group —NR^{a}R^{b}, where each of R^{a} and R^{b} is hydrogen. Additionally, “substituted amino” refers to a group —NR^{a}R^{b} wherein each of R^{a} and R^{b} individually is alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or excipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective treatment of a disorder. Treatment of a disorder may be manifested by delaying or preventing the onset or progression of the disorder, as well as the onset or progression of symptoms associated with the disorder. Treatment of a disorder may also be manifested by a decrease or elimination of symptoms, reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. Typically, to be administered in an effective dose, compounds may be administered in an amount of less than 5 mg/kg of patient weight. The compounds may be administered in an amount from less than about 1 mg/kg patient weight to less than about 100 μg/kg of patient weight, and further between about 1 μg/kg to less than 100 μg/kg of patient weight. The foregoing effective doses typically represent that amount that may be administered as a single dose, or as one or more doses that may be administered over a 24 hour period.

The compounds of this invention may be made by a variety of methods, including well-established synthetic methods. Illustrative general synthetic methods are set out below and then specific compounds of the invention are prepared in the working Examples.

In the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, herein incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art but are not described in detail here.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C- or ¹⁴C-enriched carbon are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### II. General Synthetic Methods

As will be appreciated by those skilled in the art of organic synthesis, compounds of the present invention can be made by a variety of means. Certain compounds of the present invention can be made using transformations outlined in Scheme 1 and described by Roma et al., Bioorg. Med. Chem. 8: 751-768 (2000). Thus, reaction of an alkyl malonyl chloride with a 3-aminoisoxazole derivative (i.e., a 3-aminoisoxazole appropriately substituted in either or both of the 4 and 5 positions), in the presence of an appropriate base (to neutralize the hydrochloric acid byproduct), gives a malonamide (Compound 1). Reaction of Compound 1 with phosphoryl chloride and polyphosphoric acid (PPA) provides a 5-chloro-7H-isoxazolo[2,3-a]pyrimidin-7-one derivative (substituted in either or both of the 2 and 3 positions; Compound 2). Compound 2 can then be reacted with 1,4-diazabicyclo[3.2.2]nonane to give compounds of the present invention.

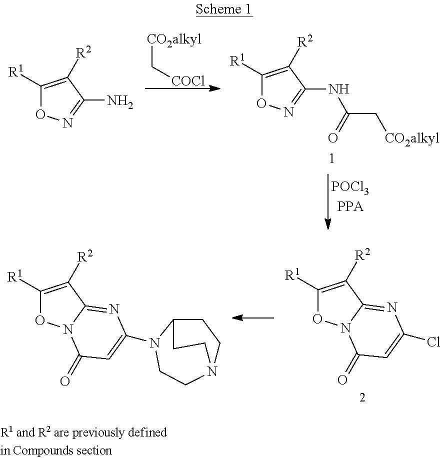

The chemistry shown in Scheme 1 is amenable to use with alkyl, aryl and fused aryl substituents on the isoxazole-derived portion (see Synthetic Examples 1-3). Also, certain intermediates shown in Scheme 1 are commercially available. For example, 5-chloro-2-methyl-7H-isoxazolo[2,3-a]pyrimidin-7-one (Compound 2, where R¹=methyl, R²=H) can be purchased from Aldrich, Enamine, and others.

As will be appreciated by those skilled in the art, the use of certain starting materials containing ancillary reactive functional groups may require additional protection/deprotection steps to prevent interference with the coupling reaction. Such protection/deprotection steps are well known in the art (for example, see T. W. Green and P. G. M. Wuts, Protective Groups in Organic Synthesis, 3^{rd} Edition, John Wiley & Sons, New York (1999)).

As will be appreciated by those skilled in the art throughout the present specification, the number and nature of substituents on rings in the compounds of the present invention will be selected so as to avoid sterically undesirable combinations.

Those skilled in the art of organic synthesis will appreciate that there exist multiple means of producing compounds of the present invention, as well as means for producing compounds of the present invention which are labeled with a radioisotope appropriate to various uses. For example, a ³H- or ¹⁴C-labeled alkyl malonyl chloride can be used as a starting material (for coupling with a suitable 3-aminoisoxazole derivative) in Scheme 1. The subsequent reactions (in Scheme 1) are amenable to retention of these “labels”, resulting in formation of an isotopically modified compound suitable for use in receptor binding and metabolism studies or as an alternative therapeutic compound.

### III. Pharmaceutical Compositions

Although it is possible to administer the compound of the present invention in the form of a bulk active chemical, it is preferred to administer the compound in the form of a pharmaceutical composition or formulation. Thus, one aspect the present invention includes pharmaceutical compositions comprising one or more compounds of Formula I and/or pharmaceutically acceptable salts thereof and one or more pharmaceutically acceptable carriers, diluents, or excipients. Another aspect of the invention provides a process for the preparation of a pharmaceutical composition including admixing one or more compounds of Formula I and/or pharmaceutically acceptable salts thereof with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation); by powder injection; or by buccal, sublingual, or intranasal absorption.

The term “intranasal delivery” or “nasal delivery” as used herein means a method for drug absorption through and within the nose. The term “buccal delivery” as used herein means a method for presenting the drug for absorption through the buccal, including inner cheek, tissue. The term “sublingual delivery” means delivery of the active agent under the tongue. Collectively, these are transmucosal delivery methods.

Drugs can be absorbed through mucosal surfaces, such as those in the nasal passage and in the oral cavity. Drug delivery via mucosal surfaces can be efficient because they lack the stratum corneum of the epidermis, a major barrier to absorption across the skin. Mucosal surfaces are also typically rich in blood supply, which can rapidly transport drugs systemically while avoiding significant degradation by first-pass hepatic metabolism.

There are three routes of absorption for drugs sprayed onto the olfactory mucosa, including by the olfactory neurons, by the supporting cells and surrounding capillary bed, and into the cerebro-spinal fluid. Absorption of drugs through the nasal mucosa tends to be rapid.

Like intranasal administration, oral transmucosal absorption is generally rapid because of the rich vascular supply to the mucosa and the lack of a stratum corneum in the epidermis. Such drug transport typically provides a rapid rise in blood concentrations, and similarly avoids the enterohepatic circulation and immediate destruction by gastric acid or partial first-pass effects of gut wall and hepatic metabolism.

Drugs typically need to have prolonged exposure to an oral mucosal surface for significant drug absorption to occur. Factors affecting drug delivery include taste, which can affect contact time, and drug ionization. Drug absorption is generally greater from the buccal or oral mucosa than from the tongue and gingiva. One limitation associated with buccal drug delivery is low flux, which often results in low drug bioavailability. Low flux may be somewhat offset by using buccal penetration enhancers, as are known in the art, to increase the flux of drugs through the mucosa.

In either of the intranasal or buccal routes, drug absorption can be delayed or prolonged, or uptake may be almost as rapid as if an intravenous bolus were administered. Because of the high permeability of the rich blood supply, the sublingual route can provide a rapid onset of action.

The intranasal, buccal, and sublingual routes can be preferred for use in treating patients who have difficulty in swallowing tablets, capsules, or other oral solids, or those who have disease-compromised intestinal absorption.

Pharmaceutical compositions may be formulated in unit dose form, or in multiple or subunit doses.

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

The compounds of the present invention may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of the compound of the present invention and one or more other therapy including chemotherapy, radiation therapy, gene therapy, or immunotherapy.

### IV. Methods/Uses

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, diabetes, metabolic syndrome, autoimmune disorders, dermatological conditions, addictions, obesity or other disorders described in further detail herein. This compound can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Yang et al., Acta Pharmacol. Sin. 30(6): 740-751 (2009), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders. Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, are believed to modulate the activity of the α7-containing NNRs without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is believed sufficient to provide the desired effects upon the disease, disorder or condition, but is believed insufficient, namely is not at a high enough level, to provide undesirable side effects.

Thus, the present invention provides the use of a compound of the present invention, or a pharmaceutically acceptable salt thereof, for use in therapy, such as a therapy described above.

In yet another aspect the present invention provides the use of a compound of the present invention, or a pharmaceutically acceptable salt thereof, in the manufacture of a medicament for use in the treatment of a CNS disorder, such as a disorder, disease or condition described hereinabove.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases.

Inflammatory conditions that can be treated or prevented by administering the compounds described herein include, but are not limited to, type II diabetes, rheumatoid arthritis, asthma, psoriasis, chronic obstructive pulmonary disease, inflammatory disease or chronic and acute inflammation, ulcerative colitis, systemic lupus erythematosus, Crohn's disease, atopic dermatitis, inflammatory bowel disease, osteoarthritis, autoimmune disease, gout, ankylosing spondylitis, transplant rejection, psoriatic arthritis, atherosclerosis, postoperative ileus, pouchitis, sarcoidosis, hypersensitivity pneumonitis, fibromyalgia, multiple sclerosis, neurodegeneration, stroke, pancreatitis, sepsis, amyotrophic lateral sclerosis, Hashimoto's thyroiditis, Addison's disease, type I diabetes, dermatomyositis, Sjogren syndrome, myasthenia gravis, Graves disease, celiac disease or sprue, uveitis, endotoxemia, gout, acute pseudogout, acute gouty arthritis, arthritis, allograft rejection, chronic transplant rejection, mononuclear-phagocyte dependent lung injury, idiopathic pulmonary fibrosis, adult respiratory distress syndrome, acute chest syndrome in sickle cell disease, irritable bowel syndrome, ulcers, acute cholangitis, aphthous stomatitis, cachexia, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis, viral pneumonitis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Neovascularization

The α7 NNR is associated with neovascularization. Inhibition of neovascularization, for example, by administering antagonists (or at certain dosages, partial agonists) of the α7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of α7 NNR.

Specific antagonism of α7 NNR-specific activity reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest. 110(4):527-36 (2002).

Representative tumor types that can be treated using the compounds described herein include NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

Alternately, treatment with α7 NNR agonists can encourage neovascularization in conditions where new vascular growth is beneficial, including those in which older vasculature has been compromised by disease (vascular diseases).

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, neuropathies such as diabetic neuropathy pain, causalgia, low back pain and deafferentation syndromes such as brachial plexus avulsion.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as lupus, disorders associated with cytokine release, cachexia secondary to infection (e.g., as occurs in AIDS, AIDS related complex and neoplasia), obesity, pemphitis, urinary incontinence, overactive bladder, diarrhea, constipation, retinal diseases, infectious diseases, myasthenia, Eaton-Lambert syndrome, hypertension, preeclampsia, osteoporosis, vasoconstriction, vasodilatation, cardiac arrhythmias, type I diabetes, type II diabetes, bulimia, anorexia, fertility disorders and sexual dysfunction, as well as those indications set forth in published PCT application WO 98/25619. The compounds of this invention can also be administered to increase the viability of stem cells in therapy, to treat convulsions such as those that are symptomatic of epilepsy, and to treat conditions such as syphillis and Creutzfeld-Jakob disease. Lastly, the compounds of this invention may be used to treat a variety of dermatological disorders, including but not limited to psoriasis, dermatitis, acne, pustulosis, vitilago, and the like.

### Diagnostic Uses

The compounds can be used in diagnostic compositions, such as probes, particularly when they are modified to include appropriate labels. The probes can be used, for example, to determine the relative number and/or function of specific receptors, particularly the α7-containing receptor subtypes. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incorporated by reference, for a disclosure of representative imaging techniques.

The radiolabeled compounds bind with high affinity to selective NNR subtypes (e.g., α7-containing) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions can be used in a method to diagnose disease in a subject, such as a human patient. The method involves administering to that patient a detectably labeled compound as described herein, and detecting the binding of that compound to selected NNR subtypes (e.g., α7-containing receptor subtypes). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the α7-containing receptor subtypes.

### Receptor Binding

The compounds of this invention can be used as reference ligands in binding assays for compounds which bind to NNR subtypes, particularly the α7-containing receptor subtypes. For this purpose the compounds of this invention are preferably labeled with a radioactive isotopic moiety such as ³H, or ¹⁴C. Examples of such binding assays are described in detail below.

### V. Synthetic Examples

### Example 1

### 5-(1,4-Diazabicyclo[3.2.2]nonan-4-yl)-2-methyl-7H-isoxazolo[2,3-a]pyrimidin-7-one hemigalactarate

1,4-Diazabicyclo[3.2.2]nonane (1.32 g, 10.5 mmol) and 5-chloro-2-methyl-7H-isoxazolo[2,3-a]pyrimidin-7-one (1.93 g, 10.5 mmol) were dissolved in anhydrous acetonitrile (52 mL). After addition of potassium carbonate (2.92 g, 20.9 mmol) and 18-crown-6 (277 mg, 1.05 mmol), the mixture was stirred and heated at reflux for 16 h. The reaction mixture was concentrated under reduced pressure. The residue was slurried in methanol (50 mL) and filtered. The filter cake was washed with methanol, and the filtrate was concentrated in vacuo. The residue was dissolved in water/TFA (10:1) and purified by preparative HPLC, using an acetonitrile/water gradient (0.05% TFA). Selected fractions were concentrated, providing 5-(1,4-diazabicyclo[3.2.2]nonan-4-yl)-2-methyl-7H-isoxazolo[2,3-a]pyrimidin-7-one trifluoroacetate (1.0 g, 25% yield), as a pale yellow oil. This material was dissolved in water (10 mL) and cooled to 0° C. in an ice bath. A solution of 5M sodium hydroxide was added drop-wise until a pH of 14 was reached. The mixture was extracted with chloroform (3×30 mL), and the combined organic extracts were dried over anhydrous sodium sulfate. The drying agent was removed by filtration, and the filtrate was concentrated in vacuo to obtain 514 mg (1.87 mmol) of 5-(1,4-diazabicyclo[3.2.2]nonan-4-yl)-2-methyl-7H-isoxazolo[2,3-a]pyrimidin-7-one free base, as a white solid (73% recovery). The free base was dissolved in methanol (2 mL) and combined with mucic (galactaric) acid (197 mg, 0.938 mmol) and water (3 mL). The mixture was sonicated for 10 min and filtered. The filtrate was concentrated to provide 582 mg of the 5-(1,4-diazabicyclo[3.2.2]nonan-4-yl)-2-methyl-7H-isoxazolo[2,3-a]pyrimidin-7-one hemigalactarate as a white solid (82% yield). ¹H NMR (400 MHz, D₂O): δ 2.06 (m, 2H), 2.24 (m, 2H), 2.41 (s, 3H), 3.40 (m, 6H), 3.84 (s, 1H, galactaric acid), 4.06 (t, 2H), 4.18 (s, 1H, galactaric acid), 4.45 (s, 1H), 5.38 (s, 1H), 6.26 (s, 1H); LCMS (m/z): 275.3 (M+1).

### Example 2

### 5-(1,4-Diazabicyclo[3.2.2]nonan-4-yl)-2-phenyl-7H-isoxazolo[2,3-a]pyrimidin-7-one

5-Phenylisoxazol-3-amine (957 mg, 5.98 mmol) was dissolved in a mixture of anhydrous dichloromethane (4 mL) and anhydrous pyridine (1.5 mL, 19 mmol). To this mixture was added, drop-wise, a solution of ethyl malonyl chloride (1.00 g, 6.64 mmol) in anhydrous dichloromethane (4 mL). The resulting warm mixture (from slight exotherm) was stirred at ambient temperature for 30 min and quenched with the addition of cold water (20 mL). Solid sodium carbonate was added until a pH of 10 was reached, and the mixture was stirred at ambient temperature for 1 hour. The organic layer was separated and the aqueous layer back-extracted with dichloromethane (4×30 mL). The combined organic extracts were passed through a phase separator column and concentrated under reduced pressure. The residue was purified via flash chromatography, utilizing a gradient of 0 to 50% ethyl acetate in hexanes, to provide ethyl 3-oxo-3-[(5-phenylisoxazol-3-yl)amino]propanoate. The entire sample was dissolved in phosphoryl chloride (1.85 mL, 30.7 mmol) and polyphosphoric acid (1.00 mL, 24.6 mmol) and heated with stirring at 110° C. for 3 h. After cooling, anhydrous ethanol (5 mL) was added to the reaction, and the mixture refluxed at 80° C. for 30 min. The reaction mixture was poured into cold water (75 mL). The precipitated solid was collected by filtration and dried in high vacuum to yield 5-chloro-2-phenylisoxazolo[2,3-a]pyrimidin-7-one as a brown solid (28% yield).

1,4-Diaza-bicyclo[3.2.2]nonane (100 mg, 0.792 mmol) and 5-chloro-2-phenyl-isoxazolo[2,3-a]pyrimidin-7-one (454 mg, 1.84 mmol) were dissolved in anhydrous acetonitrile (4 mL). After addition of potassium carbonate (221 mg, 1.58 mmol) and 18-crown-6 (21 mg, 79 pmol), the mixture was stirred and heated at reflux for 16 h. The reaction mixture was concentrated under reduced pressure. The residue was slurried in methanol (10 mL) and filtered. The filter cake was washed with methanol, and the filtrate was concentrated in vacuo. The residue was dissolved in ethanol and purified by preparative HPLC, using an acetonitrile/water gradient (0.05% TFA). Selected fractions were concentrated, providing 69.8 mg of 5-(1,4-diazabicyclo[3.2.2]non-4-yl)-2-phenyl-7H-isoxazolo[2,3-a]pyrimidin-7-one trifluoroacetate as an orange oil (20% yield). ¹H NMR (400 MHz, CD₃OD): δ 2.19 (m, 2H), 2.39 (m, 2H), 3.56 (m, 7H), 4.27 (t, 2H), 4.60 (s, 1H), 7.04 (s, 1H), 7.60 (m, 3H), 7.97 (d, 2H); LCMS (m/z): 337.5 (M+1).

### Example 3

### 2-(1,4-Diazabicyclo[3.2.2]non-4-yl)-4H-pyrimido[1,2-b][1,2]benzoxazol-4-one

1,2-Benzoxazol-3-amine (802 mg, 5.98 mmol) was dissolved in a mixture of anhydrous dichloromethane (4 mL) and anhydrous pyridine (1.5 mL, 19 mmol). To this mixture was added, drop-wise, a solution of ethyl malonyl chloride (1.00 g, 6.64 mmol) in anhydrous dichloromethane (4 mL). The resulting warm mixture (from slight exotherm) was stirred at ambient temperature for 30 min and quenched with the addition of cold water (20 mL). Solid sodium carbonate was added until a pH of 10 was reached, and the mixture was stirred at ambient temperature for 1 hour. The organic layer was separated and the aqueous layer back-extracted with dichloromethane (4×30 mL). The combined organic layers were passed through a phase separator column and concentrated under reduced pressured to yield crude ethyl 3-(1,2-benzoxazol-3-ylamino)-3-oxopropanoate. The entire sample was dissolved in phosphoryl chloride (1.85 mL, 30.7 mmol) and polyphosphoric acid (1.00 mL, 24.6 mmol) and heated with stirring at 110° C. for 4 h. After cooling, anhydrous ethanol (5 mL) was added, and the mixture was refluxed at 80° C. for 30 min. After cooling, the solution was diluted with dichloromethane and the organic layer was separated. The aqueous layer was then back-extracted with dichloromethane (4×30 mL). The combined organic layers were passed through a phase separator column and concentrated under reduced pressure. Purification via flash chromatography, utilizing a gradient of 0 to 75% ethyl acetate in hexanes, provided 2-chloropyrimido[1,2-b][1,2]benzoxazol-4-one as a white solid (489 mg, 33% yield).

1,4-Diaza-bicyclo[3.2.2]nonane (100 mg, 0.792 mmol) and 2-chloropyrimido[1,2-b][1,2]benzoxazol-4-one (489 mg, 1.84 mmol) were dissolved in anhydrous acetonitrile (4 mL). After addition of potassium carbonate (221 mg, 2.22 mmol) and 18-crown-6 (21 mg, 79 pmol), the reaction mixture was stirred and heated at reflux for 16 h. The solvent was removed under reduced pressure, and the residue was slurried in methanol (30 mL). The mixture was filtered, and the collected solid was washed with methanol. The filtrate was concentrated in vacuo. The crude material was dissolved in ethanol and purified by preparative HPLC, using an acetonitrile/water gradient (0.05% TFA). Selected fractions were concentrated, providing 56.9 mg of 2-(1,4-diazabicyclo[3.2.2]non-4-yl)-4H-pyrimido[1,2-b][1,2]benzoxazol-4-one trifluoroacetate salt as a beige solid (17% yield). ¹H NMR (400 MHz, CD₃OD): δ 2.22 (m, 2H), 2.43 (m, 2H), 3.62 (m, 7H), 4.36 (t, 2H), 4.64 (s, 1H), 7.54 (t, 1H), 7.69 (d, 1H), 7.88 (t, 1H), 8.00 (d, 1H); LCMS (m/z): 311.5 (M+1).

### Example 4

### Salt Formation

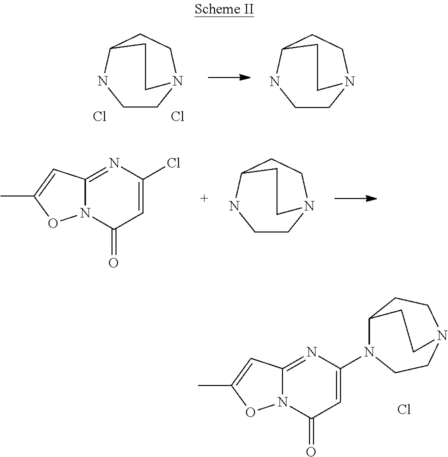

1,4-diazabicyclo[3.2.2]nonane dihydrochloride (0.81 g; 4.1 mmol) was taken up in water (4 mL; 222 mmol). The solution was cooled to 17° C. Next was added sodium hydroxide (50 mass % in H₂O; 10 mmol) and the pH was measured as ˜13+. The solution was extracted thrice with 2-methyltetrahydrofuran (15 mL total) and the combined extract solvent was removed in vacuo to yield colorless oil 1,4-diazabicyclo[3.2.2]nonane (391 mg; 3.0983 mmol; 76% Yield).

5-chloro-2-methyl-isoxazolo[2,3-a]pyrimidin-7-one was taken up in ethanol (8 mL/g) and this solution was warmed to 60° C. 1,4-diazabicyclo[3.2.2]nonane (1.0 to 2.0 equivalents); to this was added in 0.1 equivalent/hour doses until the starting pyrimidinone was consumed (monitored by UPLC/UV). The reaction was cooled to ambient temperature and filtered. The white solid was suspended in methanol (8 mL/g) at ambient temperature for 24 hours, then filtered to yield product. (40-60% Yield). ¹H NMR (D₂O) δ 6.22 (s, 1H), 5.35 (s, 1H), 4.41 (s, 1H), 4.02 (m, 2H), 3.41 (m, 6H), 2.41 (s, 3H), 2.25 (m, 2H), 2.12 (m, 2H); MS MH⁺ (C₁₄H₁₉N₄O₂) 275.2.

While the hydrochloride is exemplified, other salts may be formed using analogous procedures.

### VI. Biological Assays

### Example 5

### Characterization of Interactions at Nicotinic Acetylcholine Receptors

### Cell Lines

SH-EP1/human α4β2 (Eaton et al., 2003), SH-EP1/human α4β4 (Gentry et al., 2003), SH-EP1/α6β3β4α5 (Grinevich et al., 2005), TE671/RD and SH-SY5Y cell lines (obtained from Dr. Ron Lukas, Barrow Neurological Institute, St. Joseph's Hospital and Medical Center, Phoenix, Ariz.) were maintained in proliferative growth phase in Dulbecco's modified Eagle's medium (Gibco/BRL) with 10% horse serum (Gibco BRL), 5% fetal bovine serum (HyClone, Logan Utah), 1 mM sodium pyruvate, 4 mM L-glutamine. For maintenance of stable transfectants, the α4β2 and α4β4 cell media was supplemented with 0.25 mg/mL zeocin and 0.13 mg/mL hygromycin B. Selection was maintained for the α6β3β4α5 cells with 0.25 mg/mL of zeocin, 0.13 mg/mL of hygromycin B, 0.4 mg/mL of geneticin, and 0.2 mg/mL of blasticidin. HEK/human α7/RIC3 cells (obtained from J. Lindstrom, U. Pennsylvania, Philadelphia, Pa.) were maintained in proliferative growth phase in Dulbecco's modified Eagle's medium (Gibco/BRL) with 10% fetal bovine serum (HyClone, Logan Utah), 1 mM sodium pyruvate, 4 mM L-glutamine, 0.4 mg/mL geneticin; 0.2 mg/ml hygromycin B.

### Receptor Binding Assays

Preparation of Membranes from Rat Tissues.

Rat cortices were obtained from Analytical Biological Services, Incorporated (ABS, Wilmington, Del.). Tissues were dissected from female Sprague-Dawley rats, frozen and shipped on dry ice. Tissues were stored at −20° C. until needed for membrane preparation. Cortices from 10 rats were pooled and homogenized by Polytron (Kinematica GmbH, Switzerland) in 10 volumes (weight:volume) of ice-cold preparative buffer (KCl, 11 mM; KH₂PO₄, 6 mM; NaCl 137 mM; Na₂HPO₄ 8 mM; HEPES (free acid), 20 mM; iodoacetamide, 5 mM; EDTA, 1.5 mM; 0.1 mM PMSF pH 7.4). The resulting homogenate was centrifuged at 40,000 g for 20 minutes at 4° C. and the resulting pellet was resuspended in 20 volumes of ice-cold water. After 60-minute incubation at 4° C., a new pellet was collected by centrifugation at 40,000 g for 20 minutes at 4° C. The final pellet was resuspended in preparative buffer and stored at −20° C. On the day of the assay, tissue was thawed, centrifuged at 40,000 g for 20 minutes and then resuspended in PBS (Dulbecco's Phosphate Buffered Saline, Life Technologies, pH 7.4) to a final concentration of 2-3 mg protein/mL. Protein concentrations were determined using the Pierce BCA Protein Assay kit (Pierce Biotechnology, Rockford, Ill.), with bovine serum albumin as the standard.

Preparation of Membranes from Clonal Cell Lines.

Cells were harvested in ice-cold PBS, pH 7.4, then homogenized with a polytron (Brinkmann Instruments, Westbury, N.Y.). Homongenates were centrifuged at 40,000 g for 20 minutes (4° C.). The pellet was resuspended in PBS and protein concentration determined using the Pierce BCA Protein Assay kit (Pierce Biotechnology, Rockford, Ill.).

### Competition Binding to Receptors in Membrane Preparations.

Binding to nicotinic receptors was assayed on membranes using standard methods adapted from published procedures (Lippiello and Fernandes, 1986; Davies et al., 1999). In brief, membranes were reconstituted from frozen stocks (approximately 0.2 mg protein) and incubated for 2 h on ice in 150 ml assay buffer (PBS) in the presence of competitor compound (0.001 nM to 100 mM) and radioligand. [³H]-nicotine (L-(−)-[N-methyl-3H]-nicotine, 69.5 Ci/mmol, Perkin-Elmer Life Sciences) was used for human α4β2 binding studies. [³H]-epibatidine (52 Ci/mmol, Perkin-Elmer Life Sciences) was used for binding studies at the other receptor subtypes. Incubation was terminated by rapid filtration on a multimanifold tissue harvester (Brandel, Gaithersburg, Md.) using GF/B filters presoaked in 0.33% polyethyleneimine (w/v) to reduce non-specific binding. Filters were washed 3 times and the radioactivity retained was determined by liquid scintillation counting.

### Binding Data Analysis.

Binding data were expressed as percent total control binding. Replicates for each point were averaged and plotted against the log of drug concentration. The IC₅₀ (concentration of the compound that produces 50% inhibition of binding) was determined by least squares non-linear regression using GraphPad Prism software (GraphPAD, San Diego, Calif.). Kᵢ was calculated using the Cheng-Prusoff equation (Cheng and Prusoff, 1973).

### Example 6

### Tabular Receptor Binding Data

Compounds of Table 1, representative of the present invention, exhibited inhibition constants (Ki values) at the human α7 subtype in the range of 42 nM to 280 nM, indicating high affinity for the α7 subtype. Ki values at the α4β2 subtype are greater than 1000 nM, indicating lower affinity for the α4β2 subtype.

TABLE 1

| | Human | Human |
|---|---|---|
| | α4β2 Ki | α7 Ki |
| Structure | (nM) | (nM) |
| 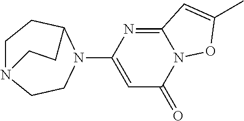 [SMILES: Cc1cc2nc(N3CCN4CCC3CC4)cc(=O)n2o1] | 3200 | 42 |
| 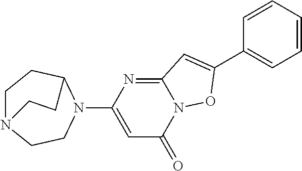 [SMILES: O=c1cc(N2CCN3CCC2CC3)nc2cc(-c3ccccc3)on12] | 1200 | 280 |
| 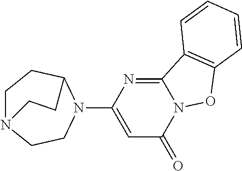 [SMILES: O=c1cc(N2CCN3CCC2CC3)nc2c3ccccc3on12] | 1200 | 55 |

### Example 7

### Ovalbumin-induced Lung Inflammation Model

Ovalbumin-induced allergic asthma is a widely used model to reproduce the airway eosinophilia, pulmonary inflammation and elevated IgE levels found during asthma. Studies can be run with or without airway hyper-responsiveness (AHR) measurements. Allergic asthma is typically triggered by allergens in the air such as pollen, mold, dust mites, etc., and is commonly characterized by reversible airway destruction, elevated levels of IgE causing mast cell activation, chronic airway inflammation, and airway hyper-responsiveness (AHR). The immunological processes involved are characterized by proliferation and activation of Th2 lymphocytes, setting off an allergic cascade.

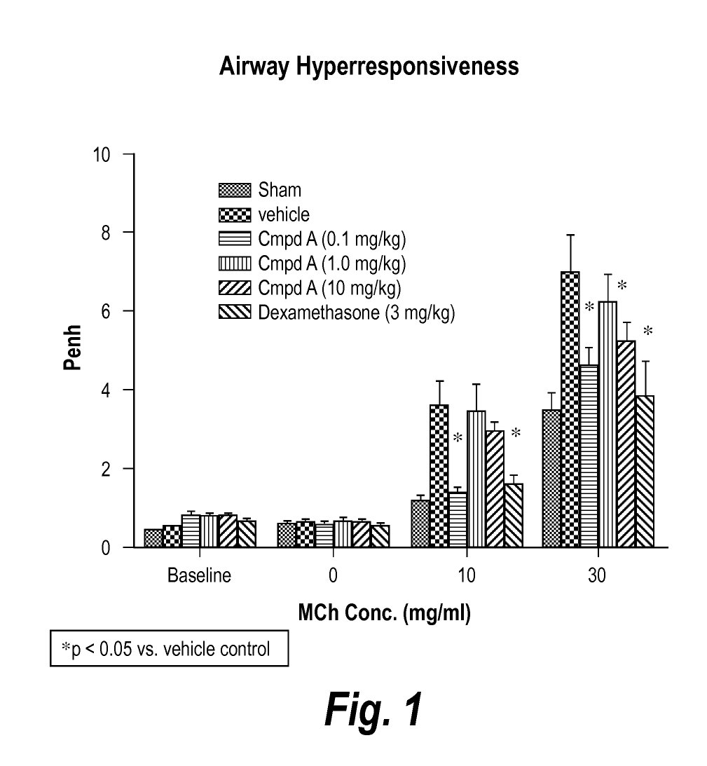

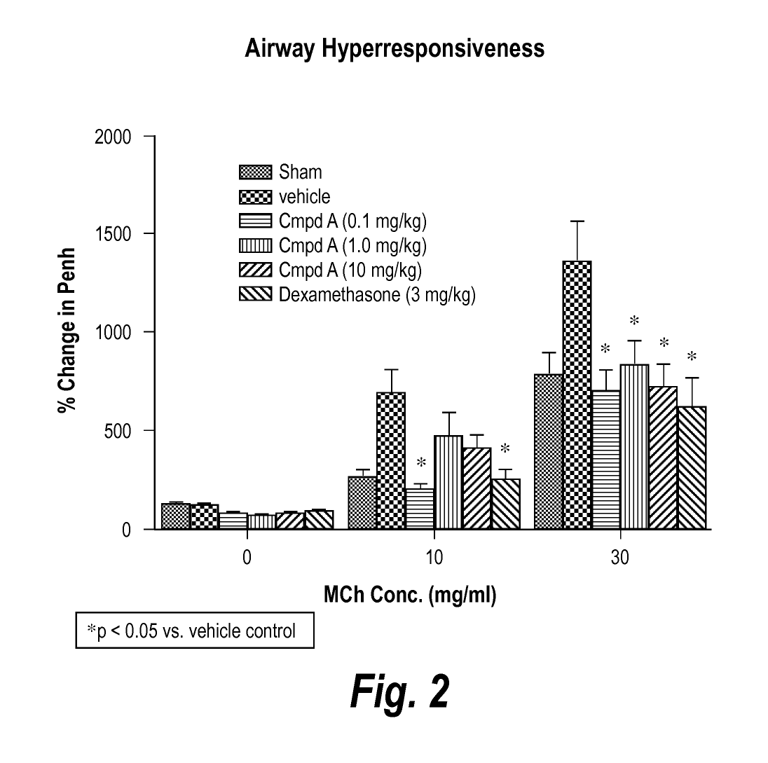

FIGS. 1 and 2 illustrate Compound A and Control compounds in the ovalbumin-induced lung inflammation model. For detailed procedures, see Hamelmann E, Schwarze J, Takeda K, Oshiba A, Larsen G L, Irvin C G, and Gelfand E W. Noninvasive measurement of airway responsiveness in allergic mice using barometric plethysmography. Am. J. Respir. Crit. Care Med 156: 766-775, 1997, herein incorporated by reference.

As shown in FIGS. 1 and 2, Compound A demonstrates statistically significant results in the present study, thereby supporting the ability of the compounds of the present invention to be useful in the treatment of, among other indications, asthma, COPD, rhinitis (especially allergic rhinitis), hypersensitivity pneumonitis (Farmer's lung), and Sarcoidosis.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

1. A compound of Formula I: 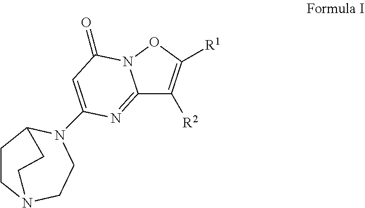
wherein:
each of R¹ and R² individually is H, C₁₋₆ alkyl, aryl, or aryl-substituted C₁₋₆ alkyl, or
R¹ and R² combine with the carbon atoms to which they are attached to form a 5- or 6-membered carbocyclic ring, either aromatic or non-aromatic,
or a pharmaceutically acceptable salt thereof.

2. A compound selected from:
5-(1,4-diazabicyclo[3.2.2]nonan-4-yl)-2-methyl-7H-isoxazolo[2,3-a]pyrimidin-7-one,
5-(1,4-diazabicyclo[3.2.2]nonan-4-yl)-2-ethyl-7H-isoxazolo[2,3-a]pyrimidin-7-one,
5-(1,4-diazabicyclo[3.2.2]nonan-4-yl)-2-benzyl-7H-isoxazolo[2,3-a]pyrimidin-7-one,
5-(1,4-diazabicyclo[3.2.2]non-4-yl)-2-phenyl-7H-isoxazolo[2,3-a]pyrimidin-7-one, and
2-(1,4-diazabicyclo[3.2.2]non-4-yl)-4H-pyrimido[1,2-b][1,2]benzoxazol-4-one,
or a pharmaceutically acceptable salt thereof.

3. A compound 5-(1,4-diazabicyclo[3.2.2]nonan-4-yl)-2-methyl-7H-isoxazolo[2,3-a]pyrimidin-7-one or a pharmaceutically acceptable salt thereof.

4. A pharmaceutical composition comprising a compound of claim 1 and one or more pharmaceutically acceptable carrier.

5. The pharmaceutical composition of claim 4, further comprising one or more additional active therapeutic agent.

6. A pharmaceutical composition comprising a compound of claim 2 and one or more pharmaceutically acceptable carrier.

7. The pharmaceutical composition of claim 6, further comprising one or more additional active therapeutic agent.

8. A method of treating a α7 mediated disorder comprising administering a compound of claim 1.

9. The method of claim 8, wherein the α7 mediated disorder is age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism, Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

10. A pharmaceutical composition comprising a compound of claim 3 and one or more pharmaceutically acceptable carrier.

11. The pharmaceutical composition of claim 10, further comprising one or more additional active therapeutic agent.

12. A method of treating a α7 mediated disorder comprising administering a compound of claim 2.

13. The method of claim 12, wherein the α7 mediated disorder is age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism, Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

14. A method of treating a α7 mediated disorder comprising administering a compound of claim 3.

15. The method of claim 14, wherein the α7 mediated disorder is age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism, Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, dyskinesias, L-dopa induced dyskinesia, tardive dyskinesia, spastic dystonia, dyskinesia, hyperkinesia, essential tremor, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.
